(19) United States
(12) Patent Application Publication
(10) Pub. No.: US 2014/0087011 A1
(43) Pub. Date: Mar. 27, 2014

(54) RyWhe / The Drink of Life

(71) Applicant: Louise M. Smith, Henderson, NV (US)

(72) Inventor: Louise M. Smith, Henderson, NV (US)

(21) Appl. No.: 13/987,806

(22) Filed: May 16, 2012

(51) Int. Cl. A61K 36/8945; A61K 36/23; A61K 36/22; A61K 36/21

(52) U.S. Cl. CPC A61K 36/8945; A61K 36/21; A61K 36/23; A61K 36/22
USPC 424/769

(57) ABSTRACT

RyWhe is a natural, raw, organic beverage. RyWhe contains 13 vitamins, the 7 major minerals, 13 trace minerals, plant proteins, fiber, 17 amino acids, healthy probiotics, living enzymes, antioxidants, flavonoids, and phytonutrients. The extraordinary combination of these all natural and organic ingredients will be made available to U.S. consumers in the form of a line of functional, ready-to-drink beverages. The inventor derived its formulations by drawing upon millennia of folk wisdom to create a line of natural functional beverages that addresses the primary health and wellness concerns of American consumers. RyWhe can be used as a vitamin supplement, which is absorbed quickly and instantly into the blood stream at 98-99%. Each beverage is carefully formulated to provide a great taste along with powerful functionality derived from natural ingredients.

RyWhe is an innovative new ready-to-drink health and wellness oriented beverage line.

RyWhe products consist of LIVING CELLS, which are exactly what make your body.

RyWhe restores, repairs, and rejuvenates. RyWhe therefore enhances longevity and quality of life. You are what you drink.

RyWhe products have all the nutrients and nutrition that your cells and body needs including vitamins and minerals, as well as alkaline elements, enzymes, probiotics, phytonutrients, antioxidants, amino acids, and flavonoids.

RyWhe is raw, organic, and natural substance in a liquid form which allows the body to absorb it quickly into the blood stream and is distributed rapidly to the cells of the body. The digestive system does not have to break it down or work hard to get it to absorb, therefore you get fast results when drinking RyWhe.

RyWhe is made with the highest quality of standards, care and love. It is authentic even in taste. To date there is no other ready to drink beverage that has what RyWhe contains. The Flavors are Beet, Sweet Potato, Carrot, and Mango.

The natural ingredients in RyWhe have been popular remedies for a range of ailments since the time of Hippocrates (460-BC-370). Hippocrates himself prescribed these ingredients for many conditions, from skin rash to ear infection.

More recently these ingredients have been used to address a wide range of health concern, including Blood Pressure, Cholesterol, Diabetes (controlling blood glucose level) Arthritis, Digestion, Sore Throat Arthritis, weight.

Each root and fruit was carefully chosen super benefits and along with a synergy of proprietary blends.

RyWhe is a complete drink, it contains all plant based vitamins, minerals and trace minerals.
Alkaline Elements
Betaine
Betacyanin
Pro-vitamin-A
Carotene A
Carotene-B
Crypto-xanthin-B
Lutein-Zeaxanthin
Lycopene
Essential Oils
Vitamins:
Vitamin A
Vitamin B (all 8)
Thiamin (B1)
Riboflavin (B2)
Niacin (B3)
Pantothenic Acid (B5)
Pyridoxine (66)
Biotin (B7)
Folate/Folic Acid (B9)
Cobalamin (B12)
Vitamin C
Vitamin D
Vitamin E
Vitamin K
Minerals:
Sulfur
Potassium
Boron
Cobalt
Calcium
Copper
Iron
Magnesium
Selenium
Phosphorous
Zinc
Iodine
Manganese
Chlorine
Nitrogenous Composites
Phytonutrients:
Flavonoids
Terpines
Sterols
Indoles
Phenols
Anti-oxidant:
Alpha, beta, gamma carotenes
Zeaxanthin
Xanthophyll
Nutrients:
Dietary Fiber
Protein
Complex Carbohydrates
Natural sugars
Enzymes
Amino Acids

However at Smith's Nutrition Products, INC we won't tell you what RyWhe will do for you, you will tell us what RyWhe does for you!

The Benefits of Beet

Beets contain vitamin A, & C, Thiamine (B1), Riboflavin (B2), Niacin (B3), Pyridoxine (B6), Calcium, Iron, phosphorus, Fat, Carbohydrates, Protein, manganese sodium, potassium, and fiber, Betaine, Alkaline Enzymes, Folic acid, Betacyanin.

Betaine is an amino acid which has anti - cancer properties. Research has also shown that beet juice can help inhibit the development of colon cancer.

Red Beet is unique for its high levels of anti-carcinogens and its very high carotenoid content. Red beets are high in carbohydrates and low in fat. Red beet is packed with antioxidant that helps the body against heart disease. The betacyanin in beets is absorbed into the blood corpuscles and can increase the oxygen-carrying ability of the blood.

Beet juice has been found to be beneficial in the treatment of Acidosis, anemia, atherosclerosis, blood pressure, cancer, constipation, dandruff, detoxification, gastric ulcer, gall bladder and kidney aliments, gout, liver ailments, and varicose veins.

Red beets help reduce serum cholesterol by 30 to 40%

Beets help in normalizing blood pressure

Beets are powerful cleanser and blood builder

Beets are good for ailments caused by toxic environment and surrounding.

Beet juice is highly alkaline which is helpful in treating acidosis

Beets help with the relief of constipation

Beets juice can also be consumed for headache, toothaches, dysentery, lumbago, skin problems, and menstrual problems.

Helps stimulate the function of liver cells and protect the liver and bile ducts.

Benefits of Carrots

In ancient times, the nutritional value of carrots lead to them being considered as medicines to cure stomach ailments, wounds liver ailments and ulcers.

Carrots have been used for thousands of years for medical purposes. They are thought by some to have healing, sedative and diuretic (reducing body fluids and bloating) properties.

The most nutritional way to eat carrots is cooking or juicing them as the cellular walls of the carrot are quite tough. Cooking or juicing carrots breaks down the cell walls.

Dr. Xiangdong Wang has carried out studies on the use of lightly cooked carrots and has found that beta-carotene is changed by the human body into retinoic acid and is widely used cancer treatment.

A natural way to build immunity against colds, flu germs, and infections is to drink carrot juice on a regular basis.

The combination of vitamins A, C and silicon found in carrots also helps promote good eye sight. According to the University of Illinois, Beta-carotene converts to vitamin A in the liver.

It is delivered to the retina via the blood stream where it is made into rhodopsin.

Rhodopsin is a purple pigment necessary for night-vision. Beta-carotene's antioxidant properties help protect against macular degeneration and the development of senile cataracts.

The chlorine and sulphur contained in carrots is a natural way to stimulate the liver to eliminate toxic waste the body. The sulphur also promotes cleansing of the digestive system and bloodstream. It is also one the main composites of insulin.

Calcium is an essential I mineral to keep the heart healthy and strong, while vitamin E is used by muscles in the body. Carrots are one of the most nutritious and healthy vegetables to eat because they are so very rich in vitamins and minerals whether eaten cooked, raw or juiced.

Carrots have a very strong antioxidant activity and aids in immune system support. They therefore aid and support mucous membrane, skin and eye health. They are also a ready source of energy and may aid to reduce the risk of heart disease, as well as calm the bowel and slow down bacterial development, while relieving you from stress fatigue and will cleanse the body.

Nutrition's, Vitamins and Minerals Found in Carrots

Complex carbohydrates, sodium, dietary fiber, natural sugars, protein, vitamin A, vitamin B1, vitamin B2, vitamin C, vitamin D vitamin E, calcium, Iron folic acid, potassium, magnesium, copper, phosphorus, sulphur, fiber, essential oils, and chlorine.

Calcium, Copper, Iron, Magnesium, Selenium, Zinc, Carotene-A, Carrotene-B, Crypto-xanthin-B, Lutein-zeaxanthin, lycopene, Enzymes, and Phytonutrients.

Benefits of Yams

Yams are one of the most nutritionally complete edible roots. They contain twice the recommended daily allowance of vitamin A, 42% of the RDA of vitamin C and four times the RDA for beta carotene. They are also exceptionally high in vitamin B6.

Yams are also high in potassium and manganese. Other important nutritional ingredients are hormones such as progesterone and an active component called diosgenin.

Beta-carotene is a carotenoid which is a precursor to vitamin A. Carotenoids have antioxidant effects and reduces the effect that free radical cells have on our bodies. They prevent oxidation and lead to repair of damaged cells. This in turn is believed to lead to reduction in the development of conditions such as atherosclerosis diabetes, heart disease and possibly colon cancer. These nutrients also have anti-inflammatory effects such as seen in asthma, osteoarthritis and rheumatoid arthritis.

Vitamin B6 is particularly helpful in patients with heart disease caused by damaged blood vessel walls. It has been strongly suggested that this damage is caused by homocysteine. Vitamin B6 is effective in the breakdown of homocysteine.

Potassium aids in controlling blood pressure through its interation?? in the maintenance of fluid balance by balancing out the sodium in the body.

Manganese is a trace mineral that helps with carbohydrate metabolism and is a cofactor in a number of enzymes important in energy production and antioxidant defenses.

The Benefits of Mango

Mango fruit is rich in pre-biotic dietary fiber, vitamins, minerals, poly-phenolic flavonoids, antioxidants and phytonutrients compounds. Several trail studies suggest that poly-phenolic antioxidant compounds in mango are known to offer protection against heart and colon cancers.

Mango fruit is an excellent source of vitamin and flavonoids like beta-carotene, alpha carotene and beta cryptoxanthin. It is also packed with potassium a mineral that is an important component of cell and body fluids that help to control heart rate and blood pressure.

One small mango provides 25% of your recommended daily allowance (RDA) for vitamin C, 67% of your RDA for vitamin A and a large amount of vitamin E & K. It is also rich in phosphorus, potassium and magnesium.

Consumption of natural fruits rich in vitamin A helps to maintain healthy mucous membranes and skin. Those fruits that are rich in carotenes are known to protect the body from lung and oral cavity cancers.

In Chinese Medicine, mangoes are used to treat anemia, bleeding gums, constipation, cough, fever nausea, sea sickness and help with weak digestion.

Some benefits include:
Fights Viruses, Cancers and Heart Disease
Boosts Libido and Enhances Sex
Lowers Cholesterol
Regulates Digestion
Fortifies Healthy Eyes

In Chinese medicine mangoes are used to treat anemia, bleeding gums, constipation, cough fever, nausea, sea and help with weak digestion.

Some benefits are fights viruses, cancers and heart disease

Boosts libido and enhances sex

lowers cholesterol

Regulates digestion

Health eyes

With the sygenic and the combination of alkaline, enzymes, and numerous of others vitamins and minerals This product becomes a much needed drink and a power house of nutrients.

Source USDA National Nutrient data base:

nutrients/Nutrition in mango Complex carbohydrates, protein, Dietary Fiber, Folates, Niacin (B3), Pantothenic acid (B5), pyridoxine (B6), Riboflavin (B2), Thiamin (B1), Vitamin-A, Vitamin-C, Vitamin-E, Vitamin-K, Sodium, Potassium,

1. Holistic health beverage that provides natural healing: including

2. Beet Flavor

3. Carrot Flavor

4. Mango Flavor

5. Sweet potato

6. Cold/Flu
